(19) United States
(12) Patent Application Publication
(10) Pub. No.: US 2024/0392211 A1
(43) Pub. Date: Nov. 28, 2024

(54) CLEANSING COMPOSITION AND METHOD

(71) Applicant: INNOSPEC LIMITED, Ellesmere, Port Cheshire (GB)

(72) Inventors: Alessandra ANDREOLI, Castiglione Delle Stiviere (IT); Massimo MOR, Castiglione Delle Stiviere (IT)

(21) Appl. No.: 18/689,704

(22) PCT Filed: Sep. 13, 2022
(86) PCT No.: PCT/GB2022/052310

§ 371 Date: Mar. 6, 2024

(30) Foreign Application Priority Data
Sep. 14, 2021 (GB) 2113089.3

(51) Int. Cl. C11D 1/14; C11D 1/28; C11D 1/29; C11D 1/66; C11D 1/83; C11D 1/90; C11D 17/00

(52) U.S. Cl. CPC C11D 1/146; C11D 1/28; C11D 1/29; C11D 1/662; C11D 1/83; C11D 1/90; C11D 17/0056; C11D 2111/14

(57) ABSTRACT

A solid cleansing composition including a hydrocarbyl sulfate and at least one of an acyl taurate and a betaine.

### FIELD OF THE INVENTION

The present invention relates to solid cleansing compositions, toilet cleansing devices, the use of solid cleansing compositions to provide cleansing to a toilet (for example as toilet blocks), and methods of providing cleansing to a toilet.

### BACKGROUND

Toilet blocks include rim blocks that are placed at the rim of a toilet bowl and cistern blocks that are placed within a toilet cistern. Toilet blocks dispense cleansing composition in the toilet bowl each time the toilet is flushed. They typically last for multiple flushes, and are thus a convenient tool for keeping a toilet clean and malodour-free for extended periods of time without having to repeatedly apply a cleansing composition to the toilet.

Currently, many toilet blocks are produced using chemicals (especially surfactants) derived from petrochemicals, such as alpha olefin sulfonates and sodium alkyl benzene sulfonates. Commonly used petrochemical-derived surfactants have good processability and foaming properties. However, the consumer market has been moving towards products that are manufactured using renewable ingredients, as renewable ingredients are considered to be more environmentally friendly than ingredients derived from petrochemicals.

Sodium lauryl sulfate is a surfactant which can be derived from renewable ingredients. However, its use in toilet blocks is limited by its poor processability and its poor foaming characteristics in the conditions typically present in toilets (e.g. hard water and water temperatures of between 1° and 20° C.). Sodium lauryl sulfate can be combined with petrochemical-based surfactants to improve the properties of the toilet block, but this decreases the overall environmental profile of the toilet block.

Therefore, there remains a need for cleansing compositions which have good processability and foaming properties that can be derived from renewable ingredients.

It is an aim of the present invention to provide an improved cleansing composition.

### SUMMARY

According to a first aspect of the present invention, there is provided a solid cleansing composition comprising a hydrocarbyl sulfate and at least one of an acyl taurate and a betaine. According to a second aspect of the present invention, there is provided a toilet cleansing device comprising the solid cleansing composition of the first aspect and an attachment element for attaching the device to a toilet.

According to a third aspect of the present invention, there is provided a use of the solid cleansing composition of the first aspect to provide cleansing to a toilet.

According to a fourth aspect of the present invention, there is provided a method of providing cleansing to a toilet, the method comprising affixing the solid cleansing composition of the first aspect to a toilet rim or placing the solid cleansing composition of the first aspect within a toilet cistern.

### DETAILED DESCRIPTION

Unless otherwise stated, the following terms used in the specification and claims have the meanings set out below.

As used herein, the term “hydrocarbyl” is used in its ordinary sense, which is well-known to those skilled in the art. Specifically, it refers to a group having a carbon atom directly attached to the remainder of the molecule and having predominantly hydrocarbon character. Examples of hydrocarbyl groups include hydrocarbon groups, i.e. aliphatic (which may be saturated or unsaturated, linear or branched, for example alkyl or alkenyl), alicyclic (for example cycloalkyl or cycloalkenyl), aromatic (for example phenyl) groups, and combinations thereof, such as aralkyl (i.e. aryl substituted alkyl) or alkaryl (i.e. alkyl substituted aryl) groups.

The term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “C₅ to C₃₀ alkyl” includes C₁₀ to C₂₅, C₅ to C₈ alkyl, pentyl, isopentyl and octyl.

The term “alkenyl” includes both straight and branched chain alkenyl groups. References to individual alkenyl groups such as “propenyl” are specific for the straight chain version only and references to individual branched chain alkenyl groups such as “isopropenyl” are specific for the branched chain version only. For example, “C₁₀ to C₂₀ alkenyl” includes C₁₂ to C₁₈ alkenyl, C₁₃ to C₁₆ alkenyl and octenyl.

Any hydrocarbyl, alkyl, alkenyl or acyl groups herein may be optionally substituted (unless stated otherwise). By “optionally substituted” groups we mean that the hydrocarbyl, alkyl, alkenyl or acyl groups are substituted or unsubstituted. Suitable substituents may include non-hydrocarbon groups provided that they do not alter the predominantly hydrocarbon nature of the hydrocarbyl, alkyl, alkenyl or acyl group. Suitable substituents may include hydroxyl, oxo, alkoxy (such as C₁ to C₄ alkoxy), amino, halo (especially fluoro and chloro), trifluoromethyl, trifluoromethoxy, cyano, alkyl (such as C₁ to C₆ alkyl), alkenyl (such as C₂ to C₆ alkenyl), alkynyl (such as C₂ to C₆ alkynyl) and aryl (such as phenyl) groups. Preferably, the hydrocarbyl, alkyl, alkenyl or acyl groups herein are unsubstituted.

References to a solid composition or component herein refer to compositions or components which are in the solid state under normal atmospheric conditions (i.e. at a pressure of 1 atmosphere and 298 K). The cleansing compositions of the present invention are solid cleansing compositions and all references herein to “cleansing compositions” or “compositions” of the invention refer to solid compositions.

As used in the specification and the appended claims, the singular forms “a”, “an,” and “the” include both singular and plural referents unless the context clearly dictates otherwise. By way of example, “a hydrocarbyl sulfate” means one hydrocarbyl sulfate or more than one hydrocarbyl sulfate.

Throughout this specification, the term “comprising” or “comprises” means including the component(s) specified but not to the exclusion of the presence of other components. The term “consisting essentially of” or “consists essentially of” means including the components specified but excluding other components except for components added for a purpose other than achieving the technical effect of the invention. The term “consisting of” or “consists of” means including the components specified but excluding other components.

Whenever appropriate, depending upon the context, the use of the term “comprises” or “comprising” may also be taken to include the meaning “consists essentially of” or “consisting essentially of”, and also may also be taken to include the meaning “consists of” or “consisting of”.

For the avoidance of doubt, where amounts of components in a composition are described in wt %, this means the weight percentage of the specified component in relation to the whole composition referred to. For example, “the hydrocarbyl sulfate may be present in the cleansing composition in an amount of from 10 to 50 wt %” means that 10 to 50 wt % of the solid cleansing composition is provided by the hydrocarbyl sulfate.

In this specification, unless otherwise indicated any amounts referred to relate to the amount of active component present in the composition. The skilled person will appreciate that commercial sources of some of the components referred to herein may include impurities, side-products and/or residual starting material. However, the amounts specified refer only to the active material and do not include any impurity, side-product, starting material or diluent that may be present.

As used herein, unless otherwise expressly specified, all numbers such as those expressing values, ranges, amounts of percentages may be read as if prefaced by the word “about”, even if the term does not expressly appear.

The term “about” as used herein when referring to a measurable value such as a parameter, an amount, a temporal duration, and the like, indicate that a value includes the standard deviation of error for the device or method being employed to determine the value. The term “about” is meant to encompass variations of +/−10% or less, +/−5% or less, or +/−0.1% or less of and from the specified value, insofar such variations are appropriate to perform in the disclosure. It is to be understood that the value to which the modifier “about” refers is itself also specifically disclosed.

The recitation of numerical ranges by endpoints includes all integer numbers and, where appropriate, fractions subsumed within that range (e.g. 1 to 5 can include 1, 2, 3, 4 when referring to, for example, a number of elements, and can also include 1.5, 2, 2.75 and 3.80, when referring to, for example, measurements). The recitation of end points also includes the end point values themselves (e.g. from 1.0 to 5.0 includes both 1.0 and 5.0). Any numerical range recited herein is intended to include all sub-ranges subsumed therein.

The optional features set out herein may be used either individually or in combination with each other where appropriate and particularly in the combinations as set out in the accompanying claims. The optional features for each exemplary embodiment of the invention, as set out herein are also applicable to any other aspects or exemplary embodiments of the invention, where appropriate. In other words, the skilled person reading this specification should consider the optional features for each aspect or embodiment of the invention as interchangeable and combinable between different aspects of the invention.

As used herein, the term “and/or,” when used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination.

According to a first aspect of the present invention, there is provided a solid cleansing composition comprising a hydrocarbyl sulfate and at least one of an acyl taurate and a betaine.

The inventors have found that cleansing compositions comprising both a hydrocarbyl sulfate and at least one of an acyl taurate and a betaine have surprisingly good processability and foaming properties, in particular compared to cleansing compositions comprising a hydrocarbyl sulfate but neither an acyl taurate nor a betaine. In addition, unlike surfactants that are commonly used in toilet blocks such as alpha olefin sulfonates and sodium alkyl benzene sulfonates, hydrocarbyl sulfates are available which are advantageously derived from renewable sources.

By “at least one of an acyl taurate and a betaine”, we mean that the cleansing composition may comprise an acyl taurate, a betaine, or both an acyl taurate and a betaine. Suitably the cleansing composition comprises an acyl taurate or a betaine.

The cleansing composition of the first aspect may comprise a hydrocarbyl sulfate and an acyl taurate.

The cleansing composition of the first aspect may comprise a hydrocarbyl sulfate and a betaine, wherein the cleansing composition has a percentage renewable carbon index of at least 90% based on the total amount of surfactants in the cleansing composition.

The solid cleansing composition of the first aspect may be in any suitable form, for example in the form of a solid bar, tablet, block, puck, stick, or sphere. The solid cleansing composition may be manufactured in a way known to a person skilled in the art, for example by compacting, pouring and moulding, or extruding.

Suitably, the solid cleansing composition is for cleansing a toilet, in particular a toilet bowl. Preferably the cleansing composition is a toilet block. Suitably the toilet block is a rim block or a cistern block.

The solid cleansing composition comprises a hydrocarbyl sulfate. The hydrocarbyl group may be unsubstituted.

The hydrocarbyl sulfate suitably comprises an alkyl sulfate, an alkenyl sulfate, or a combination thereof. The hydrocarbyl sulfate may comprise a fatty alcohol sulfate. Suitably the hydrocarbyl sulfate comprises a C₅ to C₃₀ alkyl or C₅ to C₃₀ alkenyl sulfate, such as a C₁₀ to C₂₀ alkyl or C₁₀ to C₂₀ alkenyl sulfate. The hydrocarbyl sulfate may comprise a C₅ to C₃₀ alkyl sulfate, preferably a C₁₀ to C₂₀ alkyl sulfate. The alkyl and/or alkenyl group(s) may be unsubstituted.

The hydrocarbyl sulfate may comprise a metallic hydrocarbyl sulfate and/or an amine derivative of a hydrocarbyl sulfate. By metallic hydrocarbyl sulfate we mean a hydrocarbyl sulfate comprising a metal cation. Typically, the hydrocarbyl sulfate comprises a metallic hydrocarbyl sulfate. The metallic hydrocarbyl sulfate may comprise a hydrocarbyl sulfate in which the cation is an alkali metal, such as sodium or potassium, or an alkali earth metal, such as magnesium. Preferably the metallic hydrocarbyl sulfate comprises a sodium hydrocarbyl sulfate. The amine derivative of a hydrocarbyl sulfate may comprise an ammonium hydrocarbyl sulfate, an alkyl amine hydrocarbyl sulfate, an alkanolamine hydrocarbyl sulfate, or a combination thereof.

Examples of suitable hydrocarbyl sulfates include sodium C₁₂ to C₁₆ alkyl sulfate (e.g. EMPICOL® LX series), sodium C₁₂ to C₁₈ alkyl sulfate (e.g. EMPICOL® LZ, CZ series), ammonium lauryl sulfate (e.g. EMPICOL® AL series), monoethanolamine lauryl sulfate (e.g. EMPICOL® LQ series), diethanolamine lauryl sulfate, triethanolamine lauryl sulfate (e.g. EMPICOL® TL series), monoisopropanolamine lauryl sulfate, diisopropanolamine lauryl sulfate, triisopropanolamine lauryl sulfate, magnesium lauryl sulfate, potassium lauryl sulfate, ammonium myristyl sulfate, monoethanolamine myristyl sulfate, diethanolamine myristyl sulfate, triethanolamine myristyl sulfate, sodium myristyl sulfate, ammonium cetyl sulfate, diethanolamine cetyl sulfate, sodium cetyl sulfate, sodium cetostearyl sulfate, ammonium coco-sulfate, sodium tallow sulfate, sodium oleyl sulfate, diethanolamine oleyl sulfate, sodium 2-ethylhexylsulfate (e.g. EMPICOL® 0585 series), sodium decyl sulfate (e.g. EMPICOL® 0758 series), sodium C₁₀ to C₁₂ fatty alcohol sulfate (e.g. EMPICOL® 0335 series), sodium C₈ to C₁₀ fatty alcohol sulfate, or a combination thereof. Surfactants under the name EMPICOL® are commercially available from Innospec.

Suitably, the hydrocarbyl sulfate comprises sodium C₁₂ to C₁₆ alkyl sulfate (e.g. EMPICOL® LX series), sodium C₁₂ to C₁₈ alkyl sulfate (e.g. EMPICOL® LZ, CZ series), magnesium lauryl sulfate, potassium lauryl sulfate, sodium myristyl sulfate, sodium cetyl sulfate, sodium cetostearyl sulfate, sodium tallow sulfate, sodium oleyl sulfate, sodium decyl sulfate (e.g. EMPICOL® 0758 series), sodium C₁₀ to C₁₂ fatty alcohol sulfate (e.g. EMPICOL® 0335 series), sodium C₈ to C₁₀ fatty alcohol sulfate, or a combination thereof. Preferably, the hydrocarbyl sulfate comprises sodium C₁₂ to C₁₆ alkyl sulfate (e.g. EMPICOL® LX series), sodium C₁₂ to C₁₈ alkyl sulfate (e.g. EMPICOL® LZ, CZ series), or a combination thereof.

The hydrocarbyl sulfate may be present in the cleansing composition in any suitable amount, such as in an amount of from 5 to 50 wt %, such as from 10 to 40 wt %, or even from 12 to 35 wt %, based on the total weight of the cleansing composition.

The solid cleansing composition may comprise an acyl taurate. The acyl taurate may comprise a fatty acyl taurate. The acyl taurate suitably comprises a C₅ to C₃₀ acyl taurate, such as a C₁₀ to C₂₀ acyl taurate. The acyl taurate may comprise an alkyl acyl taurate. The alkyl acyl taurate suitably comprises a C₁ to C₁₀ alkyl acyl taurate, such as a C₁ to C₆ alkyl acyl taurate, or even a C₁ to C₄ alkyl acyl taurate. The alkyl and/or acyl group(s) may be unsubstituted.

The alkyl acyl taurate suitably comprises a methyl acyl taurate. Examples of suitable alkyl acyl taurates include sodium lauroyl methyl taurate, sodium methyl myristoyl taurate, sodium methyl palmitoyl taurate, sodium methyl stearoyl taurate, sodium methyl cocoyl taurate, sodium methyl oleoyl taurate, and combinations thereof.

Preferably, the acyl taurate comprises sodium methyl oleoyl taurate.

The acyl taurate may be present in the cleansing composition in any suitable amount, such as in an amount of from 1 to 30 wt %, such as from 2 to 25 wt %, or even from 3 to 20 wt %, based on the total weight of the cleansing composition.

The solid cleansing composition may comprise a betaine. The betaine may comprise an alkyl betaine, an alkylamidopropyl betaine, an alkyl sultaine, an alkyl hydroxy sultaine, an alkylamidopropyl sultaine, an alkylamidopropyl hydroxy sultaine, or a combination thereof. Suitably, the betaine comprises an alkyl betaine, an alkylamidopropyl betaine, or a combination thereof. The betaine may comprise a C₅ to C₃₀ alkyl betaine, a C₅ to C₃₀ alkylamidopropyl betaine, a C₅ to C₃₀ alkyl sultaine, a C₅ to C₃₀ alkyl hydroxy sultaine, a C₅ to C₃₀ alkylamidopropyl sultaine, a C₅ to C₃₀ alkylamidopropyl hydroxy sultaine, or a combination thereof. The betaine may comprise a C₁₀ to C₂₀ alkyl betaine, a C₁₀ to C₂₀ alkylamidopropyl betaine, a C₁₀ to C₂₀ alkyl sultaine, a C₁₀ to C₂₀ alkyl hydroxy sultaine, a C₁₀ to C₂₀ alkylamidopropyl sultaine, a C₁₀ to C₂₀ alkylamidopropyl hydroxy sultaine, or a combination thereof. The alkyl group(s) may be unsubstituted.

Suitably, the betaine may comprise a C₅ to C₃₀ alkylamidopropyl betaine, preferably a C₁₀ to C₂₀ alkylamidopropyl betaine.

The betaine may be of the formula:

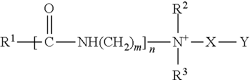

wherein R¹ is a C₅ to C₃₀ alkyl or alkenyl group, R² and R³ are each independently alkyl, hydroxyalkyl or carboxyalkyl of 1 to 6 carbon atoms; m is 2 to 4; n is 0 or 1; X is alkylene of 1 to 6 carbon atoms optionally substituted with hydroxyl; and Y is —CO₂⁻ or —SO₃⁻.

Preferably R¹ is a C₁₀ to C₂₀ alkyl or alkenyl group. R¹ may be a mixture of alkyl groups. Suitably at least half, preferably at least three quarters, of the groups R¹ by mole have 10 to 14 carbon atoms based on the total moles of betaine present.

R¹ may be a mixture of alkyl groups derived from coconut or palm kernel.

R² and R³ are preferably methyl.

The betaine may comprise an alkyl betaine of the formula:

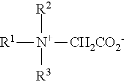

wherein R¹, R² and R³ are as defined previously.

The betaine may comprise an alkyl amido betaine of the formula:

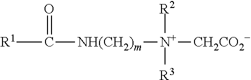

wherein R¹, R² and R³ are as defined previously; and m is 2 or 3.

The betaine may comprise a sultaine (or sulfobetaine) of the formulae:

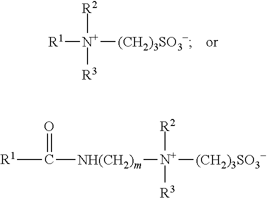

wherein R¹, R² and R³ are as defined previously; and m is 2 or 3;

or variants of these in which —(CH₂)₃SO₃⁻ is replaced by

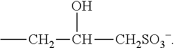

Suitable betaine surfactants include alkylamido betaine; alkyl betaine, C_{12/14} alkyldimethyl betaine; cocoamidopropylbetaine; tallow bis(hydroxyethyl) betaine; hexadecyldimethylbetaine; cocodimethylbetaine; alkyl amido propyl sulfo betaine; alkyl dimethyl amine betaine; coco amido propyl dimethyl betaine; alkyl amido propyl dimethyl amine betaine; cocamidopropyl betaine; lauryl betaine; laurylamidopropl betaine, coco amido betaine, lauryl amido betaine, alkyl amino betaine; alkyl amido betaine; coco betaine; lauryl betaine; diemethicone propyl PG-betaine; oleyl betaine; N-alkyldimethyl betaine; coco biguamide derivative, C₈ amido betaine; C₁₂ amido betaine; lauryl dimethyl betaine; alkylamide propyl betaine; amido betaine; alkyl betaine; cetyl betaine; oleamidopropyl betaine; isostearamidopropyl betaine; lauramidopropyl betaine; 2-alkyl-N-carboxymethyl-N-hydroxyethyl imidazolinium betaine; 2-alkyl-N-carboxyethyl-N-hydroxyethyl imidazolinium betaine; 2-alkyl-N-sodium carboxymethyl-N-carboxymethyl oxyethyl imidazolinium betaine; N-alkyl acid amidopropyl-N,N-dimethyl-N-(3-sulfopropyl)-ammonium-betaine; N-alkyl-N,N-dimethyl-N-(3-sulfopropyl)-ammonium-betaine; cocodimethyl betaine; apricotamidopropyl betaine; isostearamidopropyl betaine; myristamidopropyl betaine; palmitamidopropyl betaine; cocamidopropyl hydroxy sultaine; undecylenamidopropyl betaine; cocoamidosulfobetaine; alkyl amido betaine; C_{12/18} alkyl amido propyl dimethyl amine betaine; lauryldimethyl betaine; ricinol amidobetaine; tallow aminobetaine.

Suitably, the betaine comprises cocoamidopropyl betaine.

The betaine may be present in the cleansing composition in any suitable amount, such as in an amount of from 1 to 30 wt %, such as from 2 to 25 wt %, or even from 3 to 10 wt %, based on the total weight of the cleansing composition.

Suitably, the total amount of the acyl taurate and/or the betaine in the cleansing composition (whether only one of the acyl taurate or the betaine is present, or both are present) is from 1 to 30 wt %, such as from 2 to 25 wt %, or even from 3 to 20 wt %, based on the total weight of the cleansing composition.

Suitably, the combined amount of the hydrocarbyl sulfate and the at least one of the acyl taurate and the betaine in the cleansing composition is from 15 to 60 wt %, such as from 20 to 50 wt %, from 20 to 45 wt %, or even from 25 to 35 wt %, based on the total weight of the cleansing composition. Where the betaine is not present, the combined amount is of the hydrocarbyl sulfate and the acyl taurate. Where the acyl taurate is not present, the combined amount is of the hydrocarbyl sulfate and the betaine.

The cleansing composition may comprise additional components, i.e. in addition to the hydrocarbyl sulfate and the acyl taurate and/or betaine.

The cleansing composition may comprise a fatty alcohol. The fatty alcohol suitably comprises a C₁₀ to C₂₀ alkyl alcohol or a C₁₀ to C₂₀ alkenyl alcohol, such as a C₁₆ to C₁₈ alkyl alcohol or a C₁₆ to C₁₈ alkenyl alcohol. Preferably the fatty alcohol comprises a C₁₀ to C₂₀ alkyl alcohol, such as a C₁₆ to C₁₈ alkyl alcohol.

The fatty alcohol may be present in the cleansing composition in any suitable amount, such as in an amount from 1 to 10 wt %, such as from 2 to 5 wt %, based on the total weight of the cleansing composition.

The cleansing composition may comprise a hydrocarbyl glucoside and/or a glycolipid, such as a sophorolipid, a rhamnolipid, and/or a mannosylerythritol lipid.

The cleansing composition may comprise a hydrocarbyl glucoside. The hydrocarbyl glucoside suitably comprises an alkyl glucoside, an alkenyl glucoside, or a combination thereof. The hydrocarbyl glucoside may comprise a fatty alcohol glucoside. Suitably the hydrocarbyl glucoside comprises a C₅ to C₃₀ alkyl or C₅ to C₃₀ alkenyl glucoside, such as a C₁₀ to C₂₀ alkyl or C₁₀ to C₂₀ alkenyl glucoside. The hydrocarbyl glucoside may comprise a C₅ to C₃₀ alkyl glucoside, such as a C₁₀ to C₂₀ alkyl glucoside. Examples of suitable hydrocarbyl glucosides include octyl glucoside, decyl glucoside, octyldecyl glucoside, undecyl glucoside, lauryl glucoside, myristyl glucoside, cetearyl glucoside, and coco-glucoside. A suitable example of a hydrocarbyl glucoside is lauryl glucoside.

The hydrocarbyl glucoside may be present in the cleansing composition in any suitable amount, such as in an amount of from 0.1 to 5 wt %, such as from 0.25 to 2 wt %, or even from 0.5 to 1 wt %, based on the total weight of the cleansing composition.

The cleansing composition may comprise a glycolipid, such as a sophorolipid, a rhamnolipid, and/or a mannosylerythritol lipid.

The cleansing composition may comprise a hydrocarbyl ether sulfate. The hydrocarbyl ether sulfate suitably comprises an alkyl ether sulfate, an alkenyl ether sulfate, or a combination thereof. The hydrocarbyl ether sulfate may comprise a fatty alcohol ether sulfate. Suitably the hydrocarbyl ether sulfate comprises a C₅ to C₃₀ alkyl or alkenyl ether sulfate, such as a C₁₀ to C₂₀ alkyl or alkenyl ether sulfate. The hydrocarbyl ether sulfate may comprise a C₅ to C₃₀ alkyl ether sulfate, preferably a C₁₀ to C₂₀ alkyl ether sulfate. The hydrocarbyl ether sulfate may comprise a metallic hydrocarbyl ether sulfate and/or an amine derivative of a hydrocarbyl ether sulfate. By metallic hydrocarbyl ether sulfate we mean a hydrocarbyl ether sulfate comprising a metal cation. The metallic hydrocarbyl ether sulfate may comprise a hydrocarbyl ether sulfate in which the cation is an alkali metal, such as sodium or potassium, or an alkali earth metal, such as magnesium. Preferably the metallic hydrocarbyl ether sulfate comprises a sodium hydrocarbyl ether sulfate. The amine derivative of a hydrocarbyl ether sulfate may comprise an ammonium hydrocarbyl ether sulfate, an alkyl amine hydrocarbyl ether sulfate, an alkanolamine hydrocarbyl ether sulfate, or a combination thereof. A suitable example of a hydrocarbyl ether sulfate is sodium lauryl ether sulfate.

The hydrocarbyl ether sulfate may be present in the cleansing composition in any suitable amount, such as in an amount of from 0.5 to 10 wt %, such as from 1 to 5 wt %, or even from 1.5 to 2.5 wt %, based on the total weight of the cleansing composition.

The cleansing composition suitably comprises at least one of a hydrocarbyl glucoside and a hydrocarbyl ether sulfate as defined above.

The cleansing composition may comprise at least one additional ingredient selected from a pH modifier, a pH buffer, a thickener, a solubility controller, a preservative, a chelating agent, a biocide, a lime scale remover, a bleaching agent, an enzyme, a pigment, a dye, a perfume, a perfume booster, a polymer, a filler, a builder, a corrosion inhibitor, a flush regulator, a microorganism, a biofilm removal agent, a soil-adhesion inhibitor, and a combination thereof.

The cleansing composition may comprise a filler. The filler suitably comprises a carbonate salt, a sulfate salt, a halide salt, a phosphate salt, a silicate, or a combination thereof. The filler may comprise an alkali metal or alkaline earth metal carbonate salt, an alkali metal or alkaline earth metal sulfate salt, an alkali metal or alkaline earth metal halide salt, an alkali metal or alkaline earth metal phosphate salt, or a combination thereof. Suitable fillers include sodium sulfate, sodium chloride, sodium carbonate, and sodium silicate.

The filler may be present in the cleansing composition in any suitable amount, such as in an amount of at least 10 wt %, such as at least 30 wt %, or even at least 50 wt %, based on the total weight of the cleansing composition. The filler may be present in the cleansing composition in an amount of from 10 to 90 wt %, such as from 30 to 70 wt %, or even from 50 to 60 wt % based on the total weight of the cleansing composition.

The cleansing composition may comprise a builder (also known as a sequestering agent). The builder may comprise a polycarboxylic acid or a salt thereof, a carbonate salt, a phosphate salt, citric acid, a citrate salt, a silicate or a combination thereof. Suitably, the builder comprises a citrate salt, such as a tribasic citrate salt. The citrate salt may be an alkali metal citrate or an alkaline earth metal citrate. A suitable example of a citrate salt is tribasic sodium citrate. The builder may also act as a chelating agent (e.g. in the case of phosphate salts and citrate salts) and/or as a lime scale remover (e.g. in the case of citrate salts).

The builder (such as a citrate salt) may be present in the cleansing composition in any suitable amount, such as in an amount of from 0.1 to 10 wt %, such as from 0.5 to 5 wt %, or even from 0.75 to 3 wt %, based on the total weight of the cleansing composition.

The cleansing composition may comprise a microorganism. Suitably the microorganism comprises probiotics. By probiotics, we mean spores able to generate beneficial bacteria (e.g. Bacillus strains) that produce in situ highly targeted enzymes that break down fats, proteins, carbohydrates and transform them into water and carbon dioxide. Probiotics can advantageously penetrate deep into the pores of the surface to degrade a wide range of encrusted organic residues that conventional chemical-based detergents may fail to eliminate and make them easier to remove. Bacillus strains can also be used for the complete degradation of malodour sources. The microorganism, for example probiotics, may be provided in association with a builder as defined herein. For example, the cleansing composition of the first aspect may comprise tribasic sodium citrate with probiotics.

The cleansing composition of the first aspect may advantageously comprise components derived from renewable sources. Preferably the cleansing composition has a percentage renewable carbon index (% RCI) of at least 90%, suitably at least 98%, based on the total amount of surfactants in the cleansing composition. The renewable carbon index (RCI) for a component is calculated according to ISO 16128-1:2016—Annex C, wherein the number of carbon atoms derived from renewable sources is divided by the total number of carbon atoms in a component. The total RCI of the cleansing composition is a weighted average of the RCI of each carbon-containing surfactant in the cleansing composition. The % RCI is obtained by multiplying the RCI by 100. The term “surfactant” is well understood by a person skilled in the art and such a skilled person would readily understand which components of the cleansing composition of the first aspect are surfactants.

The hydrocarbyl sulfate may have a % RCI of at least 90%, suitably at least 95%, for example at least 99%, for example 100%. The hydrocarbyl sulfate may have a % RCI of from 90 to 100%, suitably from 95 to 100%, for example from 99 to 100%.

The acyl taurate may have a % RCI of at least 60%, suitably at least 70%, for example at least 80%. The acyl taurate may have a % RCI of from 60 to 100%, suitably from 70 to 100%, for example from 80 to 100%.

The betaine may have a % RCI of at least 40%, suitably at least 50%, for example at least 60%. The betaine may have a % RCI of from 40 to 100%, suitably from 50 to 100%, for example from 60 to 100%.

Solid components in the cleansing composition are suitably present in the form of powders, agglomerates, granules, needles, or combination thereof. Preferably the solid components are in the form of powders. This may be beneficial for the processability of the cleansing composition.

Suitably solid components are present in the cleansing composition in an amount of at least 80 wt %, such as at least 85 wt %, or even at least 90 wt %, based on the total weight of the cleansing composition.

The cleansing composition of the first aspect may comprise a hydrocarbyl sulfate and an acyl taurate. Suitably, the cleansing composition comprises a C₅ to C₃₀ alkyl or a C₅ to C₃₀ alkenyl sulfate and a C₅ to C₃₀ acyl taurate. Preferably, the cleansing composition comprises a C₁₀ to C₂₀ alkyl sulfate and a C₁₀ to C₂₀ acyl taurate.

The cleansing composition of the first aspect may comprise a hydrocarbyl sulfate and an acyl taurate, wherein the hydrocarbyl and acyl groups are unsubstituted. Suitably, the cleansing composition comprises a C₅ to C₃₀ alkyl or a C₅ to C₃₀ alkenyl sulfate and a C₅ to C₃₀ acyl taurate, wherein the alkyl, alkenyl and acyl groups are unsubstituted. Preferably, the cleansing composition comprises a C₁₀ to C₂₀ alkyl sulfate and a C₁₀ to C₂₀ acyl taurate, wherein the alkyl and acyl groups are unsubstituted.

For example, the cleansing composition may comprise sodium lauryl sulfate and sodium methyl oleoyl taurate.

The cleansing composition of the first aspect may comprise a hydrocarbyl sulfate having a % RCI of at least 90% and an acyl taurate having a % RCI of at least 60%, wherein the hydrocarbyl and acyl groups are unsubstituted. Suitably, the cleansing composition comprises a C₅ to C₃₀ alkyl or a C₅ to C₃₀ alkenyl sulfate having a % RCI of at least 95% and a C₅ to C₃₀ acyl taurate having a % RCI of at least 70%, wherein the alkyl, alkenyl and acyl groups are unsubstituted. Preferably, the cleansing composition comprises a C₁₀ to C₂₀ alkyl sulfate having a % RCI of at least 99% and a C₁₀ to C₂₀ acyl taurate having a % RCI of at least 80%, wherein the alkyl and acyl groups are unsubstituted.

For example, the cleansing composition may comprise sodium lauryl sulfate having a % RCI of 100% and sodium methyl oleoyl taurate having a % RCI of at least 80%.

The cleansing composition of the first aspect may comprise a hydrocarbyl sulfate and a betaine. The cleansing composition of the first aspect may comprise a hydrocarbyl sulfate and a betaine, wherein the cleansing composition has a percentage renewable carbon index of at least 90% based on the total amount of surfactants in the cleansing composition.

Suitably, the cleansing composition comprises a C₅ to C₃₀ alkyl or C₅ to C₃₀ alkenyl sulfate and a C₅ to C₃₀ alkyl betaine, a C₅ to C₃₀ alkylamidopropyl betaine, a C₅ to C₃₀ alkyl sultaine, a C₅ to C₃₀ alkyl hydroxy sultaine, a C₅ to C₃₀ alkylamidopropyl sultaine, a C₅ to C₃₀ alkylamidopropyl hydroxy sultaine, or a combination thereof. Preferably, the cleansing composition comprises a C₁₀ to C₂₀ alkyl sulfate and a C₁₀ to C₂₀ alkylamidopropyl betaine.

Suitably, the cleansing composition comprises a C₅ to C₃₀ alkyl or C₅ to C₃₀ alkenyl sulfate and a C₅ to C₃₀ alkyl betaine, a C₅ to C₃₀ alkylamidopropyl betaine, a C₅ to C₃₀ alkyl sultaine, a C₅ to C₃₀ alkyl hydroxy sultaine, a C₅ to C₃₀ alkylamidopropyl sultaine, a C₅ to C₃₀ alkylamidopropyl hydroxy sultaine, or a combination thereof, wherein the cleansing composition has a percentage renewable carbon index of at least 90% based on the total amount of surfactants in the cleansing composition. Preferably, the cleansing composition comprises a C₁₀ to C₂₀ alkyl sulfate and a C₁₀ to C₂₀ alkylamidopropyl betaine, wherein the cleansing composition has a percentage renewable carbon index of at least 90% based on the total amount of surfactants in the cleansing composition.

The cleansing composition of the first aspect may comprise a hydrocarbyl sulfate and a betaine, wherein the hydrocarbyl group is unsubstituted. The cleansing composition of the first aspect may comprise a hydrocarbyl sulfate and a betaine, wherein the hydrocarbyl group is unsubstituted and wherein the cleansing composition has a percentage renewable carbon index of at least 90% based on the total amount of surfactants in the cleansing composition. Suitably, the cleansing composition comprises a C₅ to C₃₀ alkyl or C₅ to C₃₀ alkenyl sulfate and a C₅ to C₃₀ alkyl betaine, a C₅ to C₃₀ alkylamidopropyl betaine, a C₅ to C₃₀ alkyl sultaine, a C₅ to C₃₀ alkyl hydroxy sultaine, a C₅ to C₃₀ alkylamidopropyl sultaine, a C₅ to C₃₀ alkylamidopropyl hydroxy sultaine, or a combination thereof, wherein the alkyl and alkenyl groups are unsubstituted. Preferably, the cleansing composition comprises a C₁₀ to C₂₀ alkyl sulfate and a C₁₀ to C₂₀ alkylamidopropyl betaine, wherein the alkyl groups are unsubstituted. Suitably, the cleansing composition comprises a C₅ to C₃₀ alkyl or C₅ to C₃₀ alkenyl sulfate and a C₅ to C₃₀ alkyl betaine, a C₅ to C₃₀ alkylamidopropyl betaine, a C₅ to C₃₀ alkyl sultaine, a C₅ to C₃₀ alkyl hydroxy sultaine, a C₅ to C₃₀ alkylamidopropyl sultaine, a C₅ to C₃₀ alkylamidopropyl hydroxy sultaine, or a combination thereof, wherein the alkyl and alkenyl groups are unsubstituted and wherein the cleansing composition has a percentage renewable carbon index of at least 90% based on the total amount of surfactants in the cleansing composition. Preferably, the cleansing composition comprises a C₁₀ to C₂₀ alkyl sulfate and a C₁₀ to C₂₀ alkylamidopropyl betaine, wherein the alkyl groups are unsubstituted and wherein the cleansing composition has a percentage renewable carbon index of at least 90% based on the total amount of surfactants in the cleansing composition.

For example, the cleansing composition may comprise sodium lauryl sulfate and cocoamidopropyl betaine. For example, the cleansing composition may comprise sodium lauryl sulfate and cocoamidopropyl betaine, wherein the cleansing composition has a percentage renewable carbon index of at least 90% based on the total amount of surfactants in the cleansing composition.

The cleansing composition of the first aspect may comprise a hydrocarbyl sulfate having a % RCI of at least 90% and a betaine having a % RCI of at least 40%, wherein the hydrocarbyl group is unsubstituted. Suitably, the cleansing composition comprises a C₅ to C₃₀ alkyl or C₅ to C₃₀ alkenyl sulfate having a % RCI of at least 95% and a C₅ to C₃₀ alkyl betaine having a % RCI of at least 50%, a C₅ to C₃₀ alkylamidopropyl betaine having a % RCI of at least 50%, a C₅ to C₃₀ alkyl sultaine having a % RCI of at least 50%, a C₅ to C₃₀ alkyl hydroxy sultaine having a % RCI of at least 50%, a C₅ to C₃₀ alkylamidopropyl sultaine having a % RCI of at least 50%, a C₅ to C₃₀ alkylamidopropyl hydroxy sultaine having a % RCI of at least 50%, or a combination thereof, wherein the alkyl and alkenyl groups are unsubstituted. Preferably, the cleansing composition comprises a C₁₀ to C₂₀ alkyl sulfate having a % RCI of at least 99% and a C₁₀ to C₂₀ alkylamidopropyl betaine having a % RCI of at least 60%, wherein the alkyl groups are unsubstituted.

The cleansing composition of the first aspect may comprise a hydrocarbyl sulfate having a % RCI of at least 90% and a betaine having a % RCI of at least 40%, wherein the hydrocarbyl group is unsubstituted and wherein the cleansing composition has a percentage renewable carbon index of at least 90% based on the total amount of surfactants in the cleansing composition. Suitably, the cleansing composition comprises a C₅ to C₃₀ alkyl or C₅ to C₃₀ alkenyl sulfate having a % RCI of at least 95% and a C₅ to C₃₀ alkyl betaine having a % RCI of at least 50%, a C₅ to C₃₀ alkylamidopropyl betaine having a % RCI of at least 50%, a C₅ to C₃₀ alkyl sultaine having a % RCI of at least 50%, a C₅ to C₃₀ alkyl hydroxy sultaine having a % RCI of at least 50%, a C₅ to C₃₀ alkylamidopropyl sultaine having a % RCI of at least 50%, a C₅ to C₃₀ alkylamidopropyl hydroxy sultaine having a % RCI of at least 50%, or a combination thereof, wherein the alkyl and alkenyl groups are unsubstituted and wherein the cleansing composition has a percentage renewable carbon index of at least 90% based on the total amount of surfactants in the cleansing composition. Preferably, the cleansing composition comprises a C₁₀ to C₂₀ alkyl sulfate having a % RCI of at least 99% and a C₁₀ to C₂₀ alkylamidopropyl betaine having a % RCI of at least 60%, wherein the alkyl groups are unsubstituted and wherein the cleansing composition has a percentage renewable carbon index of at least 90% based on the total amount of surfactants in the cleansing composition.

For example, the cleansing composition may comprise sodium lauryl sulfate having a % RCI of 100% and cocoamidopropyl betaine having a % RCI of at least 60%. For example, the cleansing composition may comprise sodium lauryl sulfate having a % RCI of 100% and cocoamidopropyl betaine having a % RCI of at least 60% and wherein the composition has a percentage renewable carbon index of at least 90% based on the total amount of surfactants in the composition.

According to a second aspect of the present invention, there is provided a toilet cleansing device comprising the solid cleansing composition of the first aspect and an attachment element for attaching the device to a toilet.

Suitable features of the cleansing composition in the second aspect are as defined in relation to the first aspect of the present invention.

Suitably the device is attachable to the rim of a toilet bowl. The attachment element may be in the form of an arm or hook.

The device may comprise a holder for holding the cleansing composition. The holder may be in the form of a housing such as a basket. When the toilet is flushed, water suitably contacts a portion of the cleansing composition, for example, by passing through the basket, to dissolve a portion of the cleansing composition and provide cleansing to the toilet.

According to a third aspect of the present invention, there is provided a use of the solid cleansing composition of the first aspect to provide cleansing to a toilet. For example, the cleansing composition of the first aspect may be used as a toilet block to provide cleansing to a toilet.

Suitable features of the solid cleansing composition in the third aspect are as defined in relation to the first aspect of the present invention.

According to a fourth aspect of the present invention, there is provided a method of providing cleansing to a toilet, the method comprising affixing the solid cleansing composition of the first aspect to a toilet rim or placing the solid cleansing composition of the first aspect within a toilet cistern.

Suitable features of the cleansing composition in the fourth aspect are as defined in relation to the first aspect of the present invention.

The invention will now be described with reference to the following non-limiting examples.

### EXAMPLES

### Examples 1 to 14

### Preparation of Solid Cleansing Compositions

Compositions were prepared comprising ingredients selected from those shown in Table 1.

TABLE 1

| | Appearance |
|---|---|
| | at room |
| Ingredient | temperature |
| Sodium sulfate anhydrous | solid |
| Sodium citrate tribasic dihydrate | solid |
| Sodium Citrate tribasic dihydrate with Probiotics | solid |
| Sodium carbonate anhydrous | solid |
| Empicol ® LZ/N (sodium C12-C18 alkyl sulfate | solid |
| 95% active) | |
| Empicol ® LX/B (sodium C12-C16 alkyl sulfate | solid |
| 95% active) | |
| Empiwax ® SK/BP (C16-C18 alcohol + sodium | solid |
| C12-C18 sulfate) | |
| Sodium C12-16 2EO sulfate (70% active) | liquid |
| Aquanate ® MOT 70 (sodium methyl oleoyl | solid |
| taurate 70% active) | |
| Methyl ester sulfonate | solid |
| Cocoamidopropyl betaine powder | solid |
| Empilan ® APL 50 (lauryl glucoside 50% active) | liquid |
| Perfume | liquid |

The solid ingredients shown in Tables 1 and 2 herein were weighed and added to the mixing chamber of a horizontal mixer having counter-rotating sigma blades and mixed to achieve a homogeneous mixture. The speed of the front blade was 48 rpm and the speed of the back blade was 32 rpm. The temperature of the mixing chamber was set to 25° C. The liquid ingredients shown in Tables 1 and 2 were then weighed and added to the premixed solid ingredients under mixing. The mixer was stopped once a homogeneous dough in the form of a paste or large granules had been obtained. The dough was then added to a single-screw plodder machine and extruded in the desired shape using a screw speed of 10 rpm. The temperatures of the barrel and the conical head of the plodder were set to 18.5° C. and 30° C., respectively.

### Processability

The processability of the composition was deemed to be “OK” if a dough in the form of a paste or granules was obtained after mixing and if the dough maintained its shape and did not crack or fall apart during extrusion.

### Foam Test

The foaming profile of the compositions in Table 2 was tested using the well-known Ross Miles pouring foam method (J. Ross, G. D. Miles: An Apparatus for Comparison of Foaming Properties of Soaps and Detergents, Oil & Soap, May 1941, P. 99-102.) according to ASTM D1173-53 (2001) “Foaming properties of Surface Active Agents” and UNI26001 “Cosmetic And Personal Hygiene Products—Foaming Power Determination Of Detergent Products Containing Surface Active Agents”, 1995.

The conditions of the test were as follows:

| | |
|---|---|
| Surfactant concentration | approx. 100 ppm of anionic |
| | active matter |
| Water hardness | approx. 300 ppm CaCO₃ |
| Water temperature | 15° C. |
| Repetitions | 3 |
| Measuring time of the foam | 0 sec, 60 sec, 300 sec |
| Units of foam volume | mL |

The foam was compared to a market-leading petrochemical-based toilet rim-block. The typical composition of the petrochemical-based toilet rim-block is sodium linear alkyl benzene sulfonate (85% active) 26 wt %, alpha olefin sulfonate (95% active) 18 wt %, Ceteareth-25 (100%) 8 wt %, Perfume 4 wt %, trisodium citrate dihydrate 1 wt %, sodium sulfate to 100 wt %.

The foaming of the compositions scored “OK” if they produced at least 80% of the foam volume of the benchmark (as flash foam at 0 sec and as stable foam at 300 sec).

### % RCI Calculation

The percentage renewable carbon index (% RCI) was obtained by dividing the number of carbon atoms derived from renewable sources by the total number of carbon atoms. The % RCI of the compositions was obtained by taking a weighted average of the % RCI of each of the carbon-containing surfactant ingredients. The % RCI of Empicol® LZ/N, Empicol® LX/B, Empiwax® SK/BP and Empilan® APL 50 is 100%. The % RCI of the sodium C12-16 2EO sulfate in Tables 1 and 2 was 76%. The % RCI of Aquanate® MOT 70 is 86%. The % RCI of the cocoamidopropyl betaine in Tables 1 and 2 was 66%.

TABLE 2

| Example | 1 | 2 | 3 | 4 | 5 | 6 | 7 | 8 | 9 | 10* | 11* | 12 | 13* | 14 |
|---|---|---|---|---|---|---|---|---|---|---|---|---|---|---|
| Sodium sulfate anhydrous | 54 | 54 | 55.25 | 50.25 | 57 | 54 | 54 | 55.25 | 57 | 59 | 57 | 57.25 | 62.25 | 57.25 |
| Sodium citrate tribasic dihydrate | 3 | 3 | 3 | 3 | 3 | 1 | 1 | 1 | 1 | 3 | 3 | 3 | 3 | 3 |
| Sodium citrate tribasic dihydrate with | 0 | 0 | 0 | 0 | 0 | 2 | 2 | 2 | 2 | 0 | 0 | 0 | 0 | 0 |
| Probiotics | | | | | | | | | | | | | | |
| Sodium carbonate anhydrous | 0 | 0 | 0 | 5 | 1.5 | 0 | 0 | 0 | 1.5 | 0 | 1.5 | 1.5 | 1.5 | 1.5 |
| Empicol ® LZ/N (sodium C12-C18 | 19 | 19 | 19 | 19 | 8.9 | 19 | 19 | 19 | 8.9 | 19 | 8.9 | 19 | 19 | 19 |
| alkyl sulfate 95% active) | | | | | | | | | | | | | | |
| Empicol ® LX/B (sodium C12-C16 | 10 | 10 | 10 | 10 | 4.7 | 10 | 10 | 10 | 4.7 | 10 | 4.7 | 10 | 10 | 10 |
| alkyl sulfate 95% active) | | | | | | | | | | | | | | |
| Empiwax ® SK/BP (C16-C18 alcohol + | 3.5 | 3.5 | 3.5 | 3.5 | 0 | 3.5 | 3.5 | 3.5 | 0 | 3.5 | 0 | 0 | 0 | 0 |
| sodium C12-C18 sulfate) | | | | | | | | | | | | | | |
| Sodium C12-16 2EO sulfate (70% | 2.5 | 2.5 | 0 | 0 | 0 | 2.5 | 2.5 | 0 | 0 | 2.5 | 0 | 0 | 0 | 0 |
| active) | | | | | | | | | | | | | | |
| Aquanate ® MOT 70 (sodium methyl | 5 | 0 | 5 | 5 | 20.4 | 5 | 0 | 5 | 20.4 | 0 | 0 | 5 | 0 | 2.5 |
| oleoyl taurate 70% active) | | | | | | | | | | | | | | |
| Methyl ester sulfonate | 0 | 0 | 0 | 0 | 0 | 0 | 0 | 0 | 0 | 0 | 20.4 | 0 | 0 | 0 |
| Cocoamidopropyl betaine powder | 0 | 5 | 0 | 0 | 0 | 0 | 5 | 0 | 0 | 0 | 0 | 0 | 0 | 0 |
| Empilan ® APL 50 (lauryl glucoside | 0 | 0 | 1.25 | 1.25 | 1.5 | 0 | 0 | 1.25 | 1.5 | 0 | 1.5 | 1.25 | 1.25 | 1.25 |
| 50% active) | | | | | | | | | | | | | | |
| Perfume | 3 | 3 | 3 | 3 | 3 | 3 | 3 | 3 | 3 | 3 | 3 | 3 | 3 | 3 |
| Processability (mixability, | OK | OK | OK | OK | OK | OK | OK | OK | OK | Not | Not | OK | Not | OK |
| extrudability) | | | | | | | | | | OK | OK | | OK | |
| Mixing time (min) | 60 | 29 | 90 | 90 | 40 | 60 | 29 | 90 | 40 | 120 | 180 | 40 | 180 | 40 |
| Flash foam (time 0 sec) | OK | OK | OK | OK | OK | OK | OK | OK | OK | Not | OK | OK | OK | OK |
| | | | | | | | | | | OK | | | | |
| Stable foam (time 300 sec) | OK | OK | OK | OK | OK | OK | OK | OK | OK | Not | OK | OK | Not | OK |
| | | | | | | | | | | OK | | | OK | |
| % RCI | 97.49 | 95.16 | 98.61 | 98.61 | 92.85 | 97.49 | 95.16 | 98.61 | 92.85 | 98.72 | 97.3¹ | 98 | 100 | 97.15 |

*Comparative example, ¹Estimated value.

As shown in Table 2, compositions containing a hydrocarbyl sulfate and at least one of an acyl taurate and a betaine surprisingly showed better processability and foaming properties than compositions not having either of the acyl taurate or the betaine.

### Lifespan Test

The lifespan of compositions was tested by flushing a toilet at random intervals to which a cage had been attached at the rim which contained 4×12.5 g blocks of the composition (total weight 50 g). The toilet was a Dolomite Clodia model having an approx. 10 L flush. The water hardness was approx. 300 ppm CaCO₃ and the water temperature was approx. 15° C.

The lifespan was determined as the point at which the compositions were completely used up.

Example 3 from Table 2 was compared with the market-leading petrochemical-based toilet block used in the foam test.

| | | Petrochemical-based |
|---|---|---|
| Composition | Example 3 | toilet block |
| Number of flushes | 200 | 150 |

Although a few preferred embodiments have been shown and described, it will be appreciated by those skilled in the art that various changes and modifications might be made without departing from the scope of the invention, as defined in the appended claims.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims), and/or all of the steps of any method or process so disclosed, may be combined in any combination, except combinations where at least some of such features and/or steps are mutually exclusive.

Each feature disclosed in this specification (including any accompanying claims, and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, and drawings), or to any novel one, or any novel combination, of the steps of any method or process so disclosed.

1. A solid cleansing composition comprising a hydrocarbyl sulfate and at least one of an acyl taurate and a betaine.

2. The solid cleansing composition of claim 1, comprising a hydrocarbyl sulfate and an acyl taurate.

3. The solid cleansing composition of claim 1, comprising a hydrocarbyl sulfate and a betaine, wherein the composition has a percentage renewable carbon index of at least 90% based on the total amount of surfactants in the composition.

4. The solid cleansing composition of claim 1, wherein the hydrocarbyl sulfate comprises a C₅ to C₃₀ alkyl sulfate.

5. The solid cleansing composition of claim 1, wherein the acyl taurate comprises a C₅ to C₃₀ acyl taurate.

6. The solid cleansing composition of claim 1, wherein the betaine comprises a C₅ to C₃₀ alkylamidopropyl betaine.

7. The solid cleansing composition of claim 1, wherein the combined amount of the hydrocarbyl sulfate, the acyl taurate, and the betaine in the cleansing composition is from 20 to 45 wt % based on the total weight of the cleansing composition.

8. The solid cleansing composition of claim 1, wherein the composition comprises a hydrocarbyl glucoside and/or a glycolipid.

9. The solid cleansing composition of claim 1, wherein the composition comprises a hydrocarbyl ether sulfate.

10. The solid cleansing composition of claim 9, wherein the hydrocarbyl ether sulfate comprises a C₅ to C₃₀ alkyl ether sulfate.

11. The solid cleansing composition of claim 1, wherein the composition comprises at least one additional ingredient selected from a pH modifier, a pH buffer, a thickener, a solubility controller, a preservative, a chelating agent, a biocide, a lime scale remover, a bleaching agent, an enzyme, a pigment, a dye, a perfume, a perfume booster, a polymer, a filler, a builder, a corrosion inhibitor, a flush regulator, a microorganism (such as probiotics), a biofilm removal agent, a soil-adhesion inhibitor, and a combination thereof.

12. The solid cleansing composition of claim 1, wherein the composition comprises a filler in an amount of at least 10 wt % based on the total weight of the cleansing composition.

13. The solid cleansing composition of claim 1, wherein the composition has a percentage renewable carbon index of at least 90% based on the total amount of surfactants in the composition.

14. The solid cleansing composition of claim 1, which is a toilet block.

15. The solid cleansing composition of claim 14, wherein the toilet block is a rim block or a cistern block.

16. A toilet cleansing device comprising the solid cleansing composition of claim 1 and an attachment element for attaching the device to a toilet.

17. Use of the solid cleansing composition of claim 1 to provide cleansing to a toilet.

18. A method of providing cleansing to a toilet, the method comprising affixing the solid cleansing composition of claim 1 to a toilet rim or placing the solid cleansing composition of claim 1 within a toilet cistern.
